(19) United States
(12) Patent Application Publication
(10) Pub. No.: US 2007/0248646 A1
(43) Pub. Date: Oct. 25, 2007

(54) Use of azurocidin inhibitors in prevention and treatment of ocular vascular leakage

(76) Inventors: Ali Hafezi-Moghadam, Jamaica Plain, MA (US); Dimitra Skondra, Boston, MA (US); Hyeong Gon Yu, Seoul (KR); Kousuke Noda, Cambridge, MA (US); Evangelos S. Gragoudas, Lexington, MA (US)

Correspondence Address: GOODWIN PROCTER LLP;PATENT ADMINISTRATOR, EXCHANGE PLACE, BOSTON, MA, 02109-2881, US

(21) Appl. No.: 11/706,066

(22) Filed: Feb. 13, 2007

Related U.S. Application Data
(60) Provisional application No. 60/773,889, filed on Feb. 16, 2006.

(51) Int. Cl. A61K 31/70; A61K 31/727; A61K 38/16; A61K 39/395; A61K 9/00; A61P 27/02

(52) U.S. Cl. 424/427; 424/130.1; 514/12; 514/44; 514/56

(57) ABSTRACT

Provided are methods for alleviating a symptom of an eye condition by administering to a mammal an amount of azurocidin inhibitor effective to alleviate the symptom of the eye condition.

### CROSS-REFERENCE TO RELATED APPLICATION

The present application claims the benefit of and priority to U.S. Patent Application Ser. No. 60/773,889, filed Feb. 16, 2006, the entire disclosure of which is incorporated by reference herein for all purposes.

### STATEMENT REGARDING FEDERALLY SPONSORED RESEARCH

The invention was made with funds from the NIH/NIAID Grant No. AI50775. The United States government therefore may have certain rights in the invention.

### FIELD OF THE INVENTION

The invention relates generally to methods and compositions for alleviating a symptom of an eye condition, and, more specifically, the invention relates to methods and compositions for reducing vascular permeability and/or leakage of an ocular blood vessel.

### BACKGROUND

Vascular leakage is a common feature of prominent age-related neurodegenerative diseases, such as Alzheimer's Disease and Age-Related Macular Degeneration (AMD), and may be an initial catalyst for their development. For instance, in the exudative or wet form of AMD, subretinal bleeding, fibrosis, and fluid extravasations lead to a rapid and pronounced neuronal loss (Bressler et al. (1982) AM. J. OF OPHTHALMOL. 93: 157-63). Many of the leaky vessels are newly formed, immature, and lack the sophisticated barrier function of the retina's resident vasculature (Young (1987) SURV. OPHTHALMOL. 31: 291-306). AMD is the leading cause of severe vision loss in people aged 65 and above (Bressler et al. (1988) SURV. OPHTHALMOL. 32: 375-413, Guyer et al. (1986) ARCH. OPHTHALMOL. 104: 702-05, Hyman et al. (1983) AM. J. EPIDEMIOL. 188: 816-24, Klein et al. (1982) ARCH. OPHTHALMOL. 100: 571-73, Leibowitz et al. (1980) SURV. OPHTHALMOL. 24: 335-610). AMD also is the leading cause of legal blindness in individuals older than 50 years in the Western societies (Bressler et al. (1988) supra).

It has been shown that neutrophil adhesion to the inflamed endothelium can cause vascular leakage (Kurose et al. (1994) CIRC. RES. 74: 336-43, Del Maschio et al. (1996) J. CELL. BIOL. 135: 497-510, Bolton et al. (1998) NEUROSCIENCE 86: 1245-57). One of the factors that can cause vascular leakage is vascular endothelial growth factor (VEGF), which is a hypoxia-induced angiogenic factor (Shweiki et al. (1992) NATURE 359: 843-45, Ikeda et al. (1995) J. BIOL. CHEM. 270:19761-66) and a vasopermeability factor in the retina (Senger et al. (1983) SCIENCE 219: 983-85, Dvorak et al. (1995) AM. J. PATHOL. 146:1029-39). VEGF is causally linked to the pathogenesis of diabetic retinopathy, playing an important role in promoting leukocyte-mediated changes in the retinal vasculature, and has been suggested to play a crucial role in blood retina barrier (BRB) breakdown. Within one week of experimental diabetes in rats, retinal VEGF levels increase with associated upregulation of intracellular adhesion molecule 1 (ICAM-1) in endothelial cells and its ligand, β₂-integrin, in neutrophils, leukocyte—predominantly neutrophil—adhesion, and increased retinal vascular permeability (Qaum et al. (2002) INVEST. OPHTHALMOL. VIS. SCI. 42: 2408-13, Miyamoto et al. (1999) PROC. NATL. ACAD. SCI. USA 96: 10836-41, Canas-Barouch et al. (2000) INVEST. OPHTHALMOL. VIS. SCI. 41: 1153-58). VEGF-blockade abolishes vascular leakage in the experimental model of diabetic retinopathy (Qaum et al. (2002) supra, Ishida et al. (2003) INVEST. OPHTHALMOL. VIS. SCI. 44: 2155-62, Joussen et al. (2002) AM. J. PATHOL. 160: 501-09). Furthermore, intravitreal administration of VEGF reproduces the retinal vascular changes seen in experimental diabetes, including retinal leukostasis and concomitant BRB breakdown (Ishida et al. (2003) supra). When leukocyte adhesion is inhibited by blocking ICAM-1, VEGF-induced BRB breakdown is suppressed, demonstrating the mechanistic link between leukocyte adhesion and the permeability effects of VEGF (Miyamoto et al. (2000) AM. J. PATHOL. 156: 1733-39). However, the molecular pathways involved downstream of leukocyte adhesion and how they lead to BRB breakdown is not understood.

Azurocidin (“AZ”), also known as heparin binding protein or CAP37, is an inactive protease inhibitor consisting of 225 amino acid residues. AZ is a multifunctional protein that has antimicrobial and chemotactic properties especially for monocytes (Watorek (2003) ACTA BIOCHIMICA POLONICA 50: 743-52). It is a highly glycosylated molecule of 37 kDa and is stored in neutrophils and has inflammatory properties. In vitro, AZ stimulates endothelial cells via an unknown receptor to detach and aggregate and has been shown to be involved in neutrophil-induced vascular permeability (Gautam et al. (2001) NAT. MED. 7: 1123-27). Upon neutrophil adhesion to the endothelial lining, leukocytic β₂-integrin binding with endothelial ICAM-1 causes AZ release. Azurocidin induces Ca⁺⁺-dependent cytoskeletal rearrangement and intercellular gap formation in endothelial-cell monolayers in vitro, and increases macromolecular permeability in peripheral (non-CNS) vessels in vivo (Gautam et al. (2001) supra). Moreover, AZ blockade prevents neutrophil-induced endothelial hyperpermeability, demonstrating the role of AZ in the vascular response to neutrophil adhesion during inflammation (Gautam et al. (2001) supra). However, the effect of azurocidin on vessels with barrier properties such as the blood brain barrier (BBB) or BRB, were previously unknown.

Leaky ocular vessels destroy retinal cells and cause visual loss. The main therapeutic strategies to control leakage include laser photocoagulation and photodynamic therapy (PDT) using a benzoporphyrin derivative photosensitizer. During laser photocoagulation, thermal laser light is used to heat and photocoagulate the neovasculature of the choroid. A problem associated with this approach is that the laser light must pass through the photoreceptor cells of the retina in order to photocoagulate the blood vessels in the underlying choroid. As a result, this treatment destroys the photoreceptor cells of the retina, creating blind spots with associated vision loss. During PDT, a benzoporphyrin derivative photosensitizer is administered to the individual to be treated. Once the photosensitizer accumulates in the choroidal neovasculature (CNV), non-thermal light from a laser is applied to the region to be treated, which activates the photosensitizer in that region. The activated photosensitizer generates free radicals that damage the vasculature in the vicinity of the photosensitizer (see, U.S. Pat. Nos. 5,798,349 and 6,225,303). This approach is more selective than laser photocoagulation and is less likely to result in blind spots. Under certain circumstances, this treatment has been found to restore vision in patients afflicted with the disorder (see, U.S. Pat. Nos. 5,756,541 and 5,910,510).

During clinical studies, however, it has been found that recurrence of leakage appears in at least a portion of the CNV by one to three months post-treatment. Increasing photosensitizer or light doses do not appear to prevent this recurrence, and can even lead to undesired non-selective damage to retinal vessels (Miller et al. (1999) ARCHIVES OF OPHTHALMOL. 117: 1161-73). Another avenue of investigation is to repeat the PDT procedure over prolonged periods of time. The necessity for repeated PDT treatments can nevertheless be expected to lead to cumulative damage to the retinal pigment epithelium and choriocapillaris, which may lead to progressive treatment-related vision loss. In addition, PDT can cause transient visual disturbances, injection-site adverse effects, transient photosensitivity reactions, infusion-related back pain, and vision loss. Moreover, the abnormal vessels tend to re-grow and continue to leak despite repeated treatments, which limits the therapeutic success of current approaches.

Attempts also have been made to facilitate the re-absorption of intraretinal fluid, for instance by intraocular injection of steroids. Similarly, current therapies for uveitis include corticosteroids and chemotherapeutic agents to reduce inflammation. However, the side effects of these drugs limit their use. Also, anti-VEGF agents, for example the Macugen® aptamer (see the URL address eyetk.com/science/science vegf.asp, available from Eyetech Pharmaceuticals, Inc., NY, N.Y.), a VEGF specific RNAi (see the URL address: alnylam.com/therapeutic-programs/programs.asp, available from Alnylam Pharmaceuticals, Cambridge, Mass.), and an anti-VEGF antibody or antibody fragment (see the URL address: gene.com/gene/products/information/oncology/avastin/index.jsp, available from Genentech, Inc., San Francisco, Calif.), have become available to treat AMD. However, anti-angiogenic therapy aims to halt the growth of new vessels but it is unable to regress already existing leaky vessels.

Therefore, there is a need to develop new modes of treatment of leaky ocular vessels that are more effective, more specific, and have fewer side effects than conventional methods. Further, new treatments are needed that have lower costs and long term applications.

### SUMMARY OF THE INVENTION

The importance of vascular leakage in eye conditions such as, but without limitation, diabetic retinopathy, macular edema, age-related macular degeneration, uveitis, elevated blood pressure, ischemic retinopathies, and choroidal neovascularization, indicates a need to address vascular leakage in conditions of the eye. The present invention is based, in part, upon the discovery that AZ plays a role in vascular permeability of blood vessels at the BRB and BBB. Accordingly, the invention provides a method for inhibiting AZ-induced vascular permeability and/or leakage of blood vessels at the BRB and the BBB. The method is particularly useful for treating ocular conditions, such as, but without limitation, diabetic retinopathy, macular edema, age-related macular degeneration, uveitis, elevated blood pressure, ischemic retinopathies, and choroidal neovascularization. These methods are more effective, more specific, have fewer side effects, and are potentially less invasive than current treatments. These methods also may cost less than current treatment options, with long term applications possible.

The causes of vascular leakage in age-related neurodegenerative diseases, including conditions of the eye, are not well understood. However, inflammatory processes have been implicated in BBB and BRB breakdown. Without wishing to be bound to theory, the facts that AZ affects permeability after its secretion from neutrophils adherent to the endothelium and that VEGF-induced BRB breakdown is significantly mediated through leukocyte adhesion implicate AZ as possibly playing a role in BRB breakdown induced by VEGF, downstream from leukocyte adhesion. Data provided herein elucidate this mechanism. It may be that AZ inhibition achieves its effect without interfering with leukocyte recruitment and that AZ inhibition may prevent VEGF-induced vascular leakage.

The physiology of blood vessels at the BRB and BBB is different from the physiology of blood vessels in the rest of the body. Normal blood vessels of the CNS (BBB), and similarly the blood vessels of the retina (BRB), have a unique barrier function that acts as a regulatory interface between the blood and the nervous system. The integrity of this barrier protects the nervous system from harmful blood-born molecules and cells. Various components of the CNS are necessary for the formation of the barrier. Astrocyte endfeet surround CNS microvessels and induce BBB properties in endothelial cells. These properties include the formation of high resistance tight junctions between the capillary endothelial cells that impede the passive diffusion of solutes from the blood into the extracellular space. The blood vessels at the inner BRB have a similar physiology to those at the BBB, and the outer BRB has a monolayer of retinal pigment epithelial (RPE) cells that also may create a high resistance barrier. Moreover, because of the CNS's immune privileged status, it cannot be assumed and would not be expected that a molecule, released by immune cells, would necessarily have a function in the CNS, including the retina. Thus, AZ's action, as well as an AZ inhibitor's action, at the BRB and BBB could not be predicted from activity in the peripheral vascular system.

One aspect of the invention provides a method for reducing vascular permeability of an ocular blood vessel. The method includes administering to a posterior region of a mammal's eye an amount of an azurocidin inhibitor effective to reduce vascular permeability of an ocular blood vessel located in the posterior region of the mammal's eye.

Another aspect of the invention provides a method for ameliorating a symptom of an eye condition and includes administering to a mammal an amount of azurocidin inhibitor effective to ameliorate a symptom of the eye condition. The eye condition can be, for example, diabetic retinopathy, macular edema, age-related macular degeneration, uveitis, elevated blood pressure, an ischemic retinopathy, or choroidal neovascularization. The symptom may include, for example, vascular leakage. The azurocidin inhibitor can be administered locally or systemically, and can be administered by intraocular, intravitreal, subconjunctival, or transcleral administration. The azurocidin inhibitor also can be administered by at least one of an implant, iontophoresis, and encapsulated microbubbles.

In any of the above aspects, the azurocidin inhibitor can be, for example, a Kunitz type protease inhibitor such as aprotinin, pancreatic trypsin inhibitor, WFIKKN protein, broad spectrum Kunitz type serine protease inhibitor secreted by Ancylostoma ceylanicum , potato serine protease inhibitor, trypstatin, bikunin, BbKI found in Bauhinia bauhiniodes seeds, and members of the I-α-I family of Inter-α-inhibitors; heparin; heparin-related molecules such as heparin-like glycosaminoglycans and heparin-like oligosaccharides; an antibody; an aptamer; or an siRNA.

### BRIEF DESCRIPTION OF THE DRAWINGS

The objects and features of the invention may be more fully understood by reference to the drawings described below in which:

FIG. 1 is a bar chart showing increases in retinal vascular permeability in vivo in a time dependent manner after intravitreal injection of AZ (“AZ” indicates treatment with azurocidin and “PBS” indicates treatment only with the vehicle, phosphate-buffered saline. BRB breakdown was measured 1-3 hours post-injection, 3-5 hours post-injection, and 24 hours post-injection);

FIG. 2 is a bar chart showing suppression of azurocidin-induced BRB breakdown using aprotinin (“AZ” indicates treatment with azurocidin and “AZ+aprotinin” indicates treatment with azurocidin preceded by treatment with aprotinin);

FIG. 3 is a bar chart showing suppression of VEGF-induced blood retinal barrier breakdown by AZ blockade (“VEGF” indicates treatment with VEGF, “VEGF+Aprotinin” indicates treatment with VEGF and aprotinin, “VEGF+IgG” indicates treatment with VEGF and control goat isotope IgG, “VEGF+anti-AZ” indicates treatment with goat anti-human polyclonal antibody against azurocidin preceded by treatment with VEGF);

FIG. 4A depicts a qualitative evaluation of retinal vascular permeability in VEGF-treated eyes;

FIG. 4B depicts a qualitative evaluation of reduced retinal vascular permeability in VEGF- and aprotinin-treated eyes.

FIG. 5A is a bar chart showing that aprotinin does not reduce VEGF-induced retinal leukostasis (“VEGF” indicates treatment with VEGF, “VEGF+Aprotinin” indicates treatment with VEGF and aprotinin);

FIG. 5B depicts leukocyte aggregation in control (non-VEGF treated) eyes;

FIG. 5C depicts leukocyte aggregation in VEGF-treated eyes;

FIG. 5D depicts leukocyte aggregation in VEGF- and aprotinin-treated eyes;

FIG. 6 is a graph showing changes in electrical resistance after treatment with VEGF and AZ, independently, and VEGF and AZ together. (“AZ” indicates treatment with azurocidin, “VEGF” indicates treatment with VEGF, “VEGF+AZ” indicates treatment with VEGF and AZ, and “PBS” indicates treatment only with the vehicle, phosphate-buffered saline);

FIG. 7 is a bar chart showing average CNV size in wild type mice after treatment with Aprotinin or Vehicle Control by daily subconjunctival injection seven days after laser-induced injury. (“Aprotinin” indicates treatment with aprotinin and “Control” indicates treatment with saline);

FIG. 8 is a bar chart showing leukocyte accumulation during diabetic retinopathy (“Normal” indicates leukocyte accumulation in normal rats and “Diabetic” indicates leukocyte accumulation in diabetic-model rats);

FIG. 9 is a bar chart showing that AZ-blockade suppresses retinal vascular leakage in diabetic retinopathy (“Normal” indicates retinal leakage in normal rats, “Diabetic” indicates retinal leakage in diabetic-model rats, and “Diabetic+AZ-Blockade” indicates retinal leakage in diabetic-model rats treated with aprotinin);

FIG. 10 depicts fluorescein angiograms of choroidal neovascular leakage in an experimental model of age-related macular degeneration (“AZ-Blockade” indicates treatment with aprotinin, and “Control” indicates treatment with vehicle. “7 Day Post Injury” indicates a time of 7 days after laser-induced choroidal neovascularization, and “14 Day Post Injury” indicates 14 days after laser-induced choroidal neovascularization);

FIG. 11 is a bar chart showing exacerbated choroidal neovascular lesions in ApoE−/−mice (“WT” indicates wild-type mice, and “ApoE−/−” indicates mice with an ApoE deficiency. “FA Grade 0-2A” indicates lesions graded as 0, 1, or 2A, and “FA Grade 2B” indicates lesions graded as 2B);

FIG. 12 is a bar chart showing that AZ-blockade significantly reduces retinal leukostasis in Endotoxin-Induced-Uveitis (“EIU”)(“Vehicle control” means normal rats treated with saline, “LPS-treated” means rats treated with lipopolysaccharide, and “LPS+Aprotinin” means rats treated with lipopolysaccharide and aprotinin); and

FIG. 13 is a bar chart showing that AZ-blockade significantly reduces intravitreal leukocyte accumulation twenty-four hours after EIU (“Vehicle control” means normal rats treated with saline, “LPS-treated” means rats treated with lipopolysaccharide, and “LPS+Aprotinin” means rats treated with lipopolysaccharide and aprotinin).

### DETAILED DESCRIPTION

The invention relates to methods and compositions for reducing vascular permeability and/or leakage of a blood vessel at the BBB or the BRB, including an ocular blood vessel, in a mammal, such as a human. Methods of the invention involve administering to a mammal, such as a human, an amount of an AZ inhibitor effective to ameliorate a symptom of an eye condition. Eye conditions include any that involve vascular leakage, or inflammation of the eye leading to vascular leakage, such as, diabetic retinopathy, macular edema, age-related macular degeneration, uveitis, elevated blood pressure, ischemic retinopathies, and choroidal neovascularization. In certain embodiments, an AZ inhibitor is administered to a posterior region of a mammal's eye (for example, the retina which is part of the CNS) in an amount effective to reduce vascular permeability and/or leakage of an ocular blood vessel located in the posterior region of the mammal's eye. It should be understood that although this application focuses on leakage of blood vessels at the BRB, it is contemplated that AZ inhibitors are useful to treat conditions associated with leakage of blood vessels at the BBB, such as Alzheimer's disease, utilizing the teaching herein. The link between AZ and vascular leakage in several disease states is discussed below.

The present invention provides a role for AZ in privileged (BBB or BRB bearing) vessels of the CNS (i.e., brain or retina). Normal blood vessels of the CNS have a unique barrier function (the BBB, and, in the case of the retina, the BRB) that acts as a regulatory interface between the blood and the nervous system. The integrity of this barrier is essential for the protection of the nervous system from harmful blood-born molecules and cells. Various components of the CNS are necessary for the formation of the barrier. Astrocyte endfeet surround CNS microvessels and induce BBB properties in endothelial cells. These properties include the formation of high resistance tight junctions between the capillary endothelial cells that impede the passive diffusion of solutes from the blood into the extracellular space. The blood vessels at the inner BRB have a similar physiology to those at the BBB. Additionally, the outer BRB, a monolayer of RPE cells on the Bruch's membrane, also may create a high resistance barrier.

CNS vessels are conceptually different from the vessels of the rest of the organism. One of the unique properties of the CNS is its “immune privileged status,” which is largely accomplished by the barrier function of its vasculature. The immune privileged status also means that generalized rules of the immune system may not necessarily apply to the CNS, as it has its own local cells with immune function. Because of the CNS's immune privileged status, it cannot be assumed and would not be expected that a molecule released by immune cells, such as AZ, would necessarily have a function in the CNS, including the retina. Moreover, the physiology of blood vessels at the BRB and BBB is different from the physiology of blood vessels in the rest of the body, such that activity of AZ inhibitors, or the activity of AZ itself, in peripheral vasculature is not necessarily predictive of activity on blood vessels at the BRB and BBB.

Nevertheless, the invention discerns a link between AZ, BRB and BBB, as well as vascular leakage, in several disease states. Vascular leakage is a common feature of age-related neurodegenerative diseases, such as Alzheimer's Disease and AMD, and may be an initial catalyst for their development. Age is the most important risk factor for these neurodegenerative diseases. A growing body of evidence points towards vascular and inflammatory components in the pathology of age-related neurodegenerative diseases. Specific aspects of inflammatory leukocyte endothelial interaction, over time, cause cumulative damage to CNS vessels, resulting in age-related increases in permeability. The vascular changes due to these inflammatory processes may play a role in the pathogenesis of age-related neurodegenerative diseases.

Constitutive inflammatory processes may be a cause of BBB defects during physiologic aging. This process may be accelerated under certain conditions, for instance gene defects or allelic polymorphism. ApoE has an established role in inflammatory and neurodegenerative diseases, and it has recently been discovered to play a role in BBB maintenance. The presence of the ApoE isoform, ApoE ε4, and high cholesterol are risk factors for Alzheimer's Disease and inflammatory vascular diseases (i.e., atherosclerosis), which has fostered the concept of the vascular and inflammatory nature of Alzheimer's Disease. Recent evidence also links ApoE isoforms with risk of AMD. Furthermore, patients possessing the ApoE ε4 allele are predisposed for cognitive decline after cardiac surgery and a more severe progression of Alzheimer's Disease, bolstering the evidence for inflammation in age-related neurodegeneration. Furthermore, diabetes, an inflammatory disease, is associated with an increased risk for Alzheimer's Disease, suggesting that inflammatory mechanisms in diabetes could underlie the BBB breakdown in Alzheimer's Disease. In support of this idea, long-term use of non-steroidal anti-inflammatory drugs was shown to reduce the risk of Alzheimer's Disease mortality. Though a large body of research has outlined the general mechanisms of leukocyte endothelial interaction, the specific details and consequences of leukocyte interaction with the BBB/BRB during aging remain to be elucidated.

Uveitis is one inflammatory eye disease with a symptom of vascular leakage. Uveitis can affect any part of the eye and is characterized by the accumulation of leukocytes in ocular tissues. Normally, the BRB prevents extravasation of leukocytes into the retinal tissues. During ocular inflammation there is substantial recruitment of leukocytes across the BRB. Thus, AZ inhibition may be useful to block vascular leakage in uveitis.

Diabetic retinopathy (DR) is a low-grade inflammatory disease also with a symptom of vascular leakage. In DR, leukocyte-endothelial interaction in retinal vessels precedes vascular leakage. Since the vascular leakage in DR is to a large extent responsible for the destruction of retinal cells, it is of therapeutic interest to find effective ways of preventing it. Recent evidence shows that inflammatory processes are causative of BBB/BRB breakdown. Leukocyte-endothelial interaction proceeds in a sequential manner (tethering, rolling, firm adhesion, and transmigration). Selectins mainly mediate the first steps of leukocyte-endothelial interaction. Through their lectin domain, the selectins bind to other carbohydrates presented by mucins. P-selectin is the first adhesion receptor transiently upregulated on the endothelium during inflammation, which initiates leukocyte rolling. Leukocyte accumulation in the retinal endothelium is a critical early event in the pathogenesis of diabetic retinopathy, a common cause of neurodegeneration. This process is mediated by endothelial ICAM-1 and its leukocyte ligand, CD18. The activated endothelium expresses ICAM-1, which binds to leukocyte β₂ integrins, LFA-1 (CD18CD11a) and Mac-1 (CD18CD11b), mediating firm leukocyte adhesion. Leukocytes use their integrins to extravasate through the Extra-Cellular-Matrix (ECM). β₂ integrin expression on peripheral blood neutrophils can vary during physiologic aging of leukocytes or under pathologic conditions, i.e. diabetes. When neutrophils and monocytes, two leukocyte subtypes, interact via their β₂ integrins with ICAM-1 on activated endothelium, they release azurophilic granulae. Recently, one of the protein contents of these granulae, AZ, was shown in peripheral, non-CNS vessels to cause an increase in permeability. The present invention indicates that AZ is also a potent inducer of retinal vascular leakage, which is part of the CNS vasculature.

Another important molecule involved in BRB breakdown during DR and AMD is vascular endothelial growth factor (VEGF). Recent evidence suggests that VEGF also plays a role in inflammation. It increases endothelial ICAM-1 expression, mediating increased leukocyte adhesion and BRB breakdown in DR. However, its downstream mediators remain to be investigated. The present invention indicates that AZ is a downstream effector of VEGF and should therefore be regarded as a potent regulator of BRB permeability in diseases of the eye with an inflammatory component. Thus, AZ is linked with BRB degradation, particularly with VEGF-mediated BRB degradation downstream of leukocyte adhesion.

AZ itself is an inactive serine protease, consisting of 225 amino acid residues and is a highly glycosylated molecule of 37 kDa. It is a multifunctional protein with diverse roles in host defense and inflammation. In vitro, AZ is a chemoattractant for monocytes and T-cells, and induces monocytes to differentiate into macrophages. However, it is unclear whether its chemotactic properties extend to recruitment of leukocytes to the vessels of the brain or retina. Additionally, in vitro, AZ stimulates endothelial cells via an unknown receptor to detach and aggregate.

According to the invention, in vivo application of AZ blockers reduces azurocidin or VEGF-induced BRB-permeability. Therefore, AZ blockade may find therapeutic use in ocular diseases characterized by vascular leakage, such as those with CD18/ICAM-1-mediated inflammatory leukocyte-endothelial interaction or those with VEGF-induced and leukocyte-mediated vascular leakage. So far, an important role for ICAM-1 mediated leukocyte recruitment has been proposed in DR, uveitis, macular edema, ischemic retinopathies, and choroidal neovascularization.

The serine protease inhibitor, aprotinin, binds AZ and abolishes its ability to disrupt endothelial junctions. Aprotinin, sold under the brand name Trasylol®, is used clinically to protect patients undergoing extensive surgery, i.e., cardiopulmonary bypass surgery, from leukocyte sequestration in organs and fluid loss from the vasculature. The mechanisms by which these purported benefits are achieved are proposed to be through blockade of Kallikrein and Plasmin. The use of AZ inhibitors, such as aprotinin, in the eye serves a different purpose than its use in coronary bypass surgery. In the extensive surgeries where aprotinin is currently used, acute blood loss and drop of pressure is of primary concern, not the damage that the exudates (plasma or blood cells) would cause to the extravascular cells. In contrast, in the retina the systemic effects are not of primary concern, while the damage to the local retinal cells from the leaking plasma components is important.

It is contemplated that a variety of azurocidin inhibitors may be useful in the practice of the invention. AZ inhibitors include Kunitz type protease inhibitors, such as aprotinin, pancreatic trypsin inhibitor, the WFIKKN protein, the broad spectrum Kunitz type serine protease inhibitor secreted by Ancylostoma ceylanicum, potato serine protease inhibitor, trypstatin, bikunin, BbKI found in Bauhinia bauhiniodes seeds, and members of the I-α-I family of Inter-α-inhibitors; heparin (AZ is highly glycosylated and heparin binds to and neutralizes it) and other heparin-like molecules, such as heparin-like glycosaminoglycans and heparin-like oligosaccharides; and goat anti-human azurocidin polyclonal antibody (Santa Cruz biotechnology). More generally, AZ inhibitors include proteins, peptides and derivatives thereof, including antibodies, antibody fragments, and antigen binding fragments; nucleic acids (such as DNAs, RNAs, and PNAs) and derivatives thereof, including aptamers, antisense nucleic acids, and siRNAs; and small organic and inorganic molecules.

The ability of an AZ inhibitor of interest to block AZ activity and/or reduce vascular leakage at the BRB (and/or reduce vascular permeability) can be determined, for example, using the Evans Blue technique as described in Example 1. Additionally, the transendothelial electrical resistance (TEER) technique can be used to screen potential AZ inhibitors. Human Umbilical Vein Endothelial Cells (HUVECs; Cambrex Bio Science, Baltimore, Md.) or brain microvascular endothelial cells are cultured in EGM-2 (Cambrex Bio Science) supplemented with human recombinant epidermal growth factor (hEGF), human fibroblast growth factor-basic with heparin (hFGF-B), VEGF, ascorbic acid, hydrocortisone, human recombinant insulin-like growth factor (long R3-IGF-1), heparin, gentamicin, amphotercin and 2% fetal bovine serum (FBS), at 37° C. in humidified air containing 5% CO₂. After 90% confluence is reached, the cells (2×10⁴ cells) in 100 μl of EGM-2 are brought into the upper insert of a 24-well transwell polycarbonate membrane tissue culture dish (6.5 mm diameter, 0.4 μm pore size, Corning Incorporated, Corning, N.Y.). To induce the tight junction formation between the HUVECs, 600 μl of astrocyte-conditioned medium is added in the lower chamber. Microvascular endothelial cells do not require the step of addition of astrocyte-conditioned medium. To confirm the existence of BBB properties, transwells are stained for tight-junction proteins. All measurements are performed with cells at the confluent state.

The electrical resistance across the membrane is then measured by EVOMX (World Precision Instruments, Sarasota, Fla.). Two blank inserts containing the media in the 24-well plate are used to measure background resistance. HUVECs without treatment of astrocyte-conditioned media are used as control. The values are expressed in standard units of Ω·/cm². In this model, treatment of the HUVECs (treated with astrocyte-conditioned media) or of the microvascular endothelial cells (without astrocyte-conditioned media treatment) with AZ, VEGF, TNF-alpha, or zonulin will decrease the TEER value. To the extent a candidate AZ inhibitor is useful as an AZ inhibitor, treatment of HUVECs (treated with astrocyte conditioned media and AZ, VEGF, TNF-alpha, or zonulin ) or microvascular endothelial cells (treated with AZ, VEGF, TNF-alpha, or zonulin ) with the candidate AZ inhibitor should prevent this decrease in the TEER value.

AZ inhibitors include, for example, soluble proteins that bind AZ, for example, anti-AZ antibodies, and prevent the AZ protein from binding to its cognate receptor and/or imparting its biological effect. In addition, AZ inhibitors include a nucleic acid molecule or a nucleic acid mimetic. The mode of action of these types of inhibitors may vary. For example, it is contemplated that certain nucleic acid molecules and nucleic acid mimetics may exert their effect through antisense-type technology while others may exert their effect through aptamer-type technology while others may exert their effect through RNAi technology. These same technologies (as more fully described below) can be used to reduce permeability.

In certain embodiments, useful nucleic acids include anti-AZ aptamers. The anti-AZ aptamer has a tertiary structure that permits it to bind preferentially to an AZ molecule. Methods for identifying suitable aptamers, for example, via systematic evolution of ligands by exponential enrichment (SELEX), are known in the art and are described, for example, in Ruckman et al. (1998) J. BIOL. CHEM. 273: 20556-67 and Costantino et al. (1998) J. PHARM. SCI. 87: 1412-20.

Other useful anti-AZ nucleic acid antagonists include antisense oligonucleotides. AZ gene expression, for example, can be inhibited by using nucleotide sequences complementary to a regulatory region of the AZ gene (e.g., the AZ promoter and/or a enhancer) to form triple helical structures that prevent transcription of the AZ gene in target cells. See generally, Helene (1991) ANTICANCER DRUG DES. 6(6): 569-84, Helene et al. (1992) ANN. NY ACAD. SCI. 660: 27-36; and Maher (1992) BIOESSAYS 14(12): 807-15. The antisense sequences may be modified at a base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) BIOORG. MED. CHEM. 4(1): 5-23). Peptidyl nucleic acids have been shown to hybridize specifically to DNA and RNA under conditions of low ionic strength. Additionally, RNAi techniques can be used. Double stranded RNA (dsRNA) having one strand identical (or substantially identical) to the target mRNA sequence is introduced to a cell. The dsRNA is cleaved into small interfering RNAs (siRNAs) in the cell, and the siRNAs interact with the RNA induced silencing complex to degrade the target mRNA, ultimately destroying production of a desired protein, in this case, for example, AZ. Alternatively, the siRNA can be introduced directly.

Antibodies (e.g., monoclonal or polyclonal antibodies) having sufficiently high binding specificity for the marker or target protein (for example, AZ or its receptor) can be used as AZ inhibitors. As used herein, the term “antibody” is understood to mean an intact antibody (for example, polyclonal or monoclonal antibody); an antigen binding fragment thereof, for example, a Fab, Fab′ and (Fab′)₂ fragment; and a biosynthetic antibody binding site, for example, an sFv, as described in U.S. Pat. Nos. 5,091,513; and 5,132,405; and 4,704,692. A binding moiety, for example, an antibody, is understood to bind specifically to the target, for example, AZ or its receptor, when the binding moiety has a binding affinity for the target greater than about 10⁵ M⁻¹, more preferably greater than about 10⁷ M⁻¹.

Antibodies against AZ or its receptor may be generated using standard immunological procedures well known and described in the art. See, for example, Practical Immunology, Butt, N.R., ed., Marcel Dekker, N.Y., 1984. Briefly, isolated AZ or its receptor is used to raise antibodies in a xenogeneic host, such as a mouse, goat or other suitable mammal. The AZ or its receptor is combined with a suitable adjuvant capable of enhancing antibody production in the host, and injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (for example, a cell-free emulsion).

Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Practical Immunology, Butt, W.R., ed., Marcel Dekker, New York, 1984.

The AZ inhibitor can be administered locally or systemically. Local administration can be intraocular, intravitreal, or transcleral. To limit BRB degradation (and reduce vascular permeability and/or leakage of an ocular blood vessel in the posterior region of the eye), local use of AZ inhibitors is appropriate (e.g., through an intravitreal injection, an implant, iontophoresis, and/or encapsulated microbubbles burst by ultrasound for delivering the inhibitors to the posterior region of the eye). A benefit of intravitreal injection is that it reduces systemic side-effects. For example, anaphylactic reaction, such as that seen with Trasylol®, is not expected with intravitreal injection of the drug.

The type and dosage of the AZ inhibitor administered may depend upon various factors including, for example, the age, weight, gender, and health of the individual to be treated, as well as the type and/or severity of the particular disorder to be treated. The formulations, both for veterinary and for human medical use, typically include an AZ inhibitor in association with a pharmaceutically acceptable carrier or excipient.

The carrier should be acceptable in the sense of being compatible with the other ingredients of the formulations and not deleterious to the recipient. Pharmaceutically acceptable carriers, in this regard, are intended to include any and all solvents, dispersion media, coatings, isotonic and absorption delaying agents, and the like, that are compatible with pharmaceutical administration. The use of such media and agents for pharmaceutically active substances is known in the art. Supplementary active compounds (identified or designed according to the invention and/or known in the art) also can be incorporated into the formulations. The formulations may conveniently be presented in dosage unit form and may be prepared by any of the methods well known in the art of pharmacy/microbiology. In general, some formulations are prepared by bringing the active molecule into association with a liquid carrier or a finely divided solid carrier or both, and then, if necessary, shaping the product into the desired formulation.

A pharmaceutical composition of the invention should be formulated to be compatible with its intended route of administration. Examples of routes of administration include local or systemic routes. Local routes include, for example, topical application to the eye, or intraorbital, periorbital, sub-tenons, intravitreal, subconjunctival (for example, a subconjunctival implant), transscleral delivery, pos terior implantation, iontophoresis, and encapsulated microbubbles that are burst by ultrasound. Intravitreal injection, implants, iontophoresis, and/or liposomal bubbles may be useful for delivering pharmaceutical compositions of the invention to the posterior portion of the eye to affect posterior blood vessels. Systemic routes include, for example, oral or parenteral routes, or alternatively via intramuscular, intravenous, subcutaneous, intradermal, inhalation, transdermal (topical), transmucosal, and rectal routes.

Formulations suitable for oral or parenteral administration may be in the form of discrete units such as capsules, gelatin capsules, sachets, tablets, troches, or lozenges, each containing a predetermined amount of the antibiotic; a powder or granular composition; a solution or a suspension in an aqueous liquid or non-aqueous liquid; or an oil-in-water emulsion or a water-in-oil emulsion. Formulations suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). Sterile injectable solutions can be prepared by incorporating the active compound in the required amount in an appropriate solvent with one or a combination of ingredients enumerated above, as required, followed by filter sterilization. Formulations suitable for intra-articular administration may be in the form of a sterile aqueous preparation of the drug which may be in microcrystalline form, for example, in the form of an aqueous microcrystalline suspension. Liposomal formulations or biodegradable polymer systems may also be used to present the drug for both intra-articular and ophthalmic administration. Biodegradable or non-biodegradable implants that are associated with and release the drug may be used. Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. To the extent a particular active compound is too acidic or basic for use, basic or acidic co-substances or buffering systems, can be incorporated into the formulation.

Administration may be provided as a periodic bolus (for example, intravenously or intravitreally) or as continuous infusion from an internal reservoir (for example, from an implant disposed at an intra- or extra-ocular location (see, U.S. Pat. Nos. 5,443,505 and 5,766,242)) or from an external reservoir (for example, from an intravenous bag). The AZ inhibitor may be administered locally, for example, by continuous release from a sustained release drug delivery device immobilized to an inner wall of the eye or via targeted transscleral controlled release into the choroid (see, for example, PCT/US00/00207, PCT/US02/14279, Ambati et al. (2000) INVEST. OPHTHALMOL. VIS. SCI. 41:1181-1185, and Ambati et al. (2000) INVEST. OPHTHALMOL. VIS. SCI. 41:1186-1191). A variety of devices suitable for administering an AZ inhibitor locally to the inside of the eye are known in the art. See, for example, U.S. Pat. Nos. 6,251,090, 6,299,895, 6,416,777, 6,413,540, and 6,375,972, and PCT/US00/28187. Additionally, encapsulated microbubbles, filled with, for example, air or a higher weight molecular gas, ranging in size from 1 -10 μm in diameter, and having, for example, albumin shells can be used for AZ inhibitor delivery. At specific ultrasound wavelengths, these hard-shelled microspheres are brought to resonance such that they burst and deliver their contents. See, for example, Allen et al. (2001), IEEE, 48(2):409-18.

The AZ inhibitor also may be administered in a pharmaceutically acceptable carrier or vehicle so that administration does not otherwise adversely affect the recipient's electrolyte and/or volume balance. The carrier may comprise, for example, physiologic saline or other buffer system.

In addition, it is contemplated that the AZ inhibitor may be formulated so as to permit release of the AZ inhibitor over a prolonged period of time. A release system can include a matrix of a biodegradable material or a material which releases the incorporated AZ inhibitor by diffusion. The AZ inhibitor can be homogeneously or heterogeneously distributed within the release system. A variety of release systems may be useful in the practice of the invention, however, the choice of the appropriate system will depend upon rate of release required by a particular drug regime. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that AZ inhibitors having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

One of the primary vehicles currently being developed for the delivery of ocular pharmacological agents is the poly(lactide-co-glycolide) microsphere for intraocular injection. The microspheres are composed of a polymer of lactic acid and glycolic acid, which are structured to form hollow spheres. These spheres can be approximately 15-30 μm in diameter and can be loaded with a variety of compounds varying in size from simple molecules to high molecular weight proteins such as antibodies. The biocompatibility of these microspheres is well established (see, Sintzel et al. (1996) EUR. J. PHARM. BIOPHARM. 42: 358-372), and microspheres have been used to deliver a wide variety of pharmacological agents in numerous biological systems. After injection, poly(lactide-co-glycolide) microspheres are hydrolyzed by the surrounding tissues, which cause the release of the contents of the microspheres (Zhu et al. (2000) NAT. BIOTECH. 18: 52-57). As will be appreciated, the in vivo half-life of a microsphere can be adjusted depending on the specific needs of the system.

In therapeutic use for treating ocular disorders, the active ingredients (i.e., AZ inhibitors) typically are administered intravitreally, subconjunctivally, orally, parenterally and/or topically to provide a therapeutically effective amount in the individual, for example, an amount of the active ingredient, for example, in the blood and/or tissue, sufficient to prevent or diminish ocular vascular leakage. Generally, an effective amount of dosage of active molecule will be in the range of from about 0.1 mg/kg to about 100 mg/kg, optionally from about 1.0 mg/kg to about 50 mg/kg of body weight/day. The amount administered likely will depend on such variables as the type and extent of disease or indication to be treated, the overall health status of the particular patient, the relative biological efficacy of the compound delivered, the formulation of the drug, the presence and types of excipients in the formulation, and the route of administration. Also, it is understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

The invention is illustrated further by reference to the following non-limiting examples.

### EXAMPLES

### Example 1

### Characterization of Azurocidin as a Permeability Factor in the Retina: Involvement in Leukocyte-Mediated and VEGF-Induced Blood Retina Barrier Breakdown

The purpose of this experiment is to demonstrate that azurocidin (AZ) increases retinal vascular permeability in vivo and that AZ is involved in VEGF-induced BRB breakdown, downstream from leukocyte adhesion. These results provide support that specific targeting of AZ can serve as a treatment for vascular leakage in diseases in which leakage is an issue, such as, but without limitation, AMD and diabetic retinopathy.

This study employed Brown Norway rats, AZ (1 μg, 10 μg, 20 μg), PBS, aprotinin, VEGF₁₆₄, and polyclonal anti-AZ antibody (1 μg). Briefly, Brown Norway rats received intravitreal injections of AZ (1 μg, 10 μg and 20 μg) in one eye and PBS in the contralateral eye. BRB breakdown was quantified using the Evans Blue (EB) technique at 1 hour, 3 hours, and 24 hours after intravitreal injection of AZ or PBS. To assess whether AZ induced retinal leukostasis, firm leukocyte adhesion was quantified at 3 hours and 24 hours post AZ injection (10 μg) using the ex vivo conconavalin leukostasis assay. To block AZ, rats were treated with a single intravenous injection of aprotinin (30,000KIU), prior to the intravitreal injections. To investigate whether AZ was involved in VEGF-induced BRB breakdown, rats received intravitreal injections of 50 ng recombinant mouse VEGF₁₆₄ in one eye and PBS in the other eye and were treated intravenously with aprotinin (50,000KIU every eight hours) or polyclonal anti-AZ antibody (1 μg). BRB breakdown and retinal leukostasis were quantified 24 hours after VEGF injection.

Materials and Methods

Animals

All animal experiments adhered to the ARVO Statement for the Use of Animals in Ophthalmic and Vision Research and were approved by the Animal Care Committee of the Massachusetts Eye and Ear Infirmary. The rats were fed standard laboratory chow and allowed free access to water in an air-conditioned room with a 12-hour light-dark cycle until they were used for the experiments. Male Brown Norway rats, weighing 200-300 g were used for the experiments.

Animals were anesthetized with intramuscular xylazine hydrochloride (6 mg/kg; Phoenix Pharmaceutical, St. Joseph, Mo.) and ketamine hydrochloride (40 mg/kg; Parke-Davis, Morris Plains, N.J.). Intravitreal injections were performed by inserting a 31-gauge needle (Hamilton Company) into the vitreous 1 mm posterior to the corneal limbus. Insertion and infusion were directly viewed through an operating microscope, in order to prevent injury to the lens or the retina. Eyes that exhibited signs of damage to the lens or retina were excluded from the experiments. Intravenous injections were performed through the tail vein with a 27 Gauge needle butterfly under anesthesia.

Administration of Aurocidin and Aprotinin

Rats received intravitreous injections of 5 μl of sterile phosphate-buffered saline (PBS) containing 20 μg human neutrophil azurocidin (Athens Research and Biotechology, Atlanta, Ga.) in one eye and 5 μl of sterile phosphate-buffered saline (PBS) in the contralateral eye. The retinas were analyzed 45 minutes, 3 hours, and 24 hours after azurocidin injection.

In a second group, the rats were additionally treated intravenously with the azurocidin inhibitor, aprotinin (Trasylol®, Bayer Pharmaceuticals). 30,000KIU of aprotinin (3 ml of Trasylol®) was administered intravenously though the tail vein one hour before the intravitreal injection of azurocidin. The retinas were analyzed one hour after azurocidin administration using the Evans Blue technique.

Administration of VEGF, Aprotinin and Anti-Azurocidin Antibody

Rats received intravitreous injections of 5 μl of sterile phosphate-buffered saline (PBS) containing 50 ng VEGF₁₆₄ (R&D Systems, Minneapolis, Minn.) in one eye and 5 μl of sterile phosphate-buffered saline (PBS) in the contralateral eye. The retinas were analyzed for leakage 24 hours after VEGF injection.

In a second group, the rats were additionally treated intravenously with aprotinin (Trasylol®, Bayer Pharmaceuticals). 50,000KIU of aprotinin (5 ml of Trasylol®) was administered intravenously through the tail vein 1 hour before, as well as 8 hours and 16 hours after, the intravitreal injection of VEGF. The retinas were analyzed 24 hours after VEGF administration.

In a third group, rats were treated with intravitreous injections of 1 μg of goat anti-human azurocidin polyclonal antibody (5 μl of a 200 μg/ml antibody solution, Santa Cruz biotechnology) or control goat isotype IgG (R&D system) in the VEGF injected eye 6 hours after VEGF administration.

Blood Retina Barrier Breakdown Measurement with Evans Blue (EB) Technique

Retinal vascular permeability was quantified as previously described (Qaum et al. (2002) supra, Xu et al. (2001) INVEST. OPHTHALMOL. VIS. SCI. 42; 789-94). After the animals were deeply anesthetized, Evans blue dye (Sigma) dissolved in normal saline (30 mg/ml) was injected through the tail vein over 10 seconds at a dosage of 45 mg/kg. Blood samples were obtained from the left ventricle, just before perfusion at 2 hours, to obtain the time-average Evans blue dye plasma concentration. These blood samples were centrifuged at 12,000 rpm for 15 minutes and diluted to 1/10,000th of their initial concentration in formamide (Sigma). The background-subtracted absorbance was determined by measuring each sample at 620 nm (the absorbance maximum for Evans blue dye in formamide) and 740 nm (the absorbance minimum) with a spectrophotometer. The concentration of dye in the plasma was calculated from a standard curve of Evans blue dye in formamide. After the dye had circulated for 2 hours, the chest cavity was opened, and the rats were perfused through the left ventricle with 1% paraformaldehyde in citrate buffer (0.05 M, pH 3.5) at a physiological pressure of 120 mm Hg. The retinas were then carefully dissected under an operating microscope. After measurement of the retinal weight, Evans blue dye was extracted by incubating each retina in 0.180 ml of formamide for 18 hours at 70° C. The extract was ultracentrifuged at a speed of 14,000 rpm for 60 minutes at 25° C. Sixty microliters of the supernatant was used for spectrophotometric measurement at 620 nm and 740 nm. The background-subtracted absorbance was determined by measuring each sample at 620 nm (the absorbance maximum for Evans blue dye in formamide) and 740 nm (the absorbance minimum). The concentration of dye in the extracts was calculated from a standard curve of Evans blue dye in formamide. Blood-retinal barrier breakdown was calculated as previously described (Qaum et al (2002) supra, Xu et al. (2001) supra). Briefly, as mentioned, the optical density of the formamide containing tissue-extracted Evans Blue dye and that of the animals' plasma were measured at two different wavelengths (620 and 740 nm). The values were then inserted into the following equation.

$$\frac{( {OD}_{retina ( 620 nm )} - {OD}_{retina ( 740 nm )} ) \times Volume of formamide ( µl )}{\begin{matrix}( {OD}_{plasma ( 620 nm )} - {OD}_{plasma ( 740 nm )} ) / \\ Dry {Weight}_{retina} ( mg ) \times {time}_{EB \text{-} circulation} ( h )\end{matrix}}$$

Results were expressed as a percentage of the value in control eyes. As used through each of Examples 1 -12, the units for results from the EB technique are μl plasma/g retina/h.
Ex Vivo Quantification of Retinal Leukostasis

Deep anesthesia was induced with xylazine and ketamine as above. The chest cavity was carefully opened and the left ventricle was entered with a 14-gauge perfusion cannula fixed to a vessel clamp; care was taken to avoid ventricular obstruction. The right atrium was opened with a 12-gauge needle to achieve outflow. With the heart providing the motive force, 250 ml/kg PBS (roughly 30 ml of PBS quickly) was perfused to clear erythrocytes and non-sticking leukocytes, followed by perfusion with FITC-coupled Concanavalin A lectin (20 μg/ml in PBS, pH 7.4, total concentration 5 mg/kg) (Vector Labs, Burlingame, Calif.). The latter stained adherent leukocytes and the vascular endothelium. Lectin staining was followed by PBS perfusion (30 ml quickly).

The retinas were flat-mounted in a water-based fluorescence-anti-fading medium (Southern Biotechnology, Birmingham, Ala.) and imaged via fluorescence microscopy (Leica upright microscope, Hamamatsu digital high sensitivity camera, openlab imaging software). The total number of adherent leukocytes per retina was counted in a masked fashion.

Qualitative Evaluation and Visualization of Retinal Vascular Permeability

Retinal vascular permeability was documented in a histological manner by intravenous injection of 20-kDa FITC conjugated dextran (50 mg/Kg, Sigma Aldrich). Rats were sacrificed 30 minutes later with intracardiac perfusion of 4% paraformaldehyde to fix the dextran conjugate in the tissues. The retinas were carefully dissected and flat mounted in anti-fading medium (Vector Laboratories). Flat-mount retinas were examined by fluorescent microscopy. Digital color enhancement (green) was equally applied to all images for improved visualization of fluorescence.

Results

Azurocidin Increases Retinal Vascular Permeability In Vivo

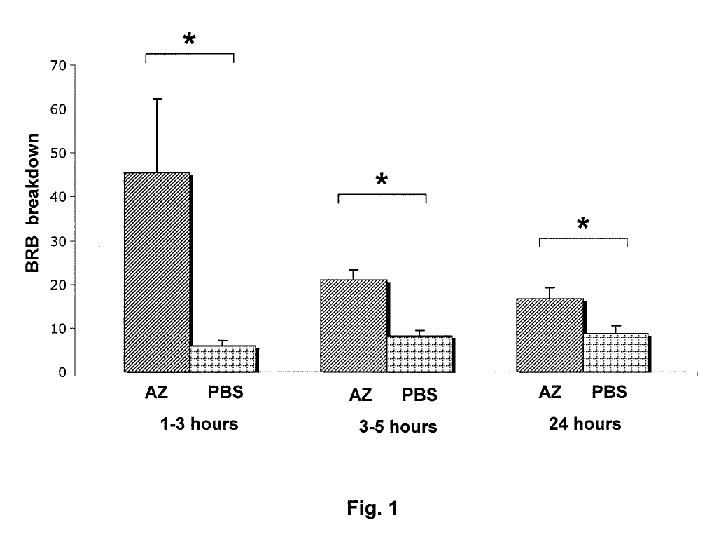

To investigate whether azurocidin is able to mediate leakage of vessels with neurovascular barrier properties, the effect of intravitreal administration of azurocidin on retinal vascular permeability was assessed by the Evans Blue technique. Intravitreal injection of 20 μg of azurocidin was given in one eye and vehicle (PBS) alone was given to the contralateral eye. Azurocidin increased retinal vascular permeability in vivo in a time-dependent manner, with a peak one to three hours post administration (FIG. 1). Intravitreal injection of 20 μg of azurocidin induced a 7.7-fold increase in Evans Blue retinal leakage compared to control eyes approximately one to three hours after injection (45±17 vs 5.8±1.4, p=0.03, n=8 animals), a 2.7-fold increase 3 to 5 hours post injection (21±2.3 vs 8.3±1.4, P=0.0006, n=11 animals), and a 1.7-fold increase 24 hours post-injection (16.5±2.8 vs 8.7±1.8, P=0.04, n=6 animals) (FIG. 1).

Aprotinin Suppresses Azurocidin-Induced BRB Leakage

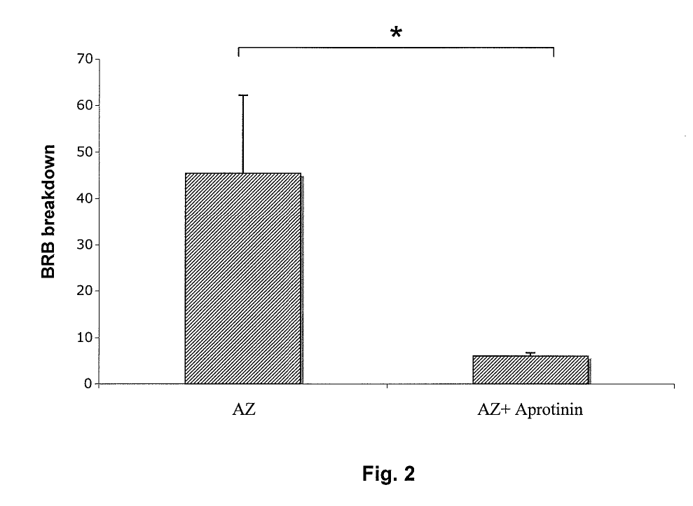

To investigate whether the protease inhibitor, aprotinin, can prevent azurocidin-induced BRB breakdown, rats were treated with a single intravenous injection of aprotinin 1 hour before intravitreal injection of azurocidin or PBS. Retinal vascular permeability was quantified with the Evans Blue technique 1 hour later. Treatment of the animals with aprotinin blocked AZ-induced leakage by greater than 98% (45±17 vs 6±0.64, P=0.03), while aprotinin treatment alone did not affect basal levels of retinal vascular leakage (FIG. 2).

Suppression of VEGF-Induced BRB Breakdown Using Aprotinin or Antibody against Azurocidin

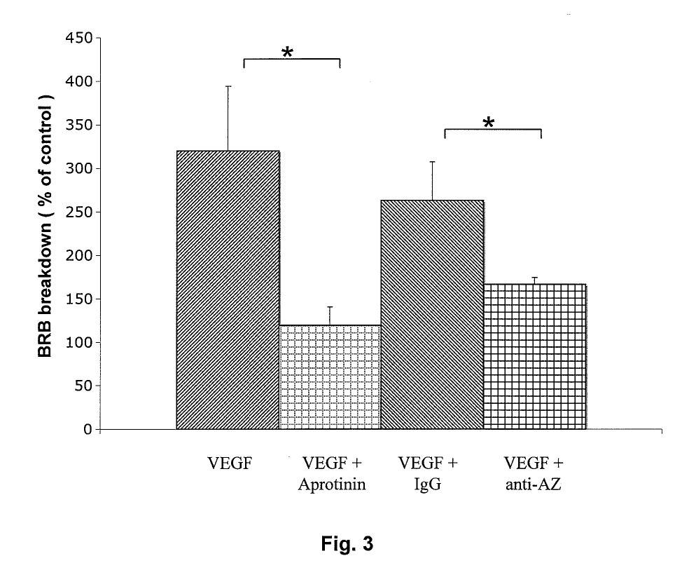

In line with previous reports, intravitreal injection of 50 ng VEGF induced a 3.2±0.74 fold increase in retinal vascular leakage 24 hours after intravitreal injection compared to the PBS injected eye (29.7±7.5 vs 9.6±1.4, P=0.02 ). BRB breakdown was measured using the Evans Blue assay. The VEGF-induced blood retinal barrier breakdown was suppressed by 95% to 1.1±0.21 fold (6.2±1.5 vs 5.4±1.4) by intravenous administration of aprotinin (320%±74 vs 110%±21, P=0.03). Aprotinin did not reduce the basal levels of retinal Evans Blue leakage (FIG. 3). Treatment with antibody against azurocidin 6 hours after VEGF administration decreased blood retina barrier breakdown by 68% to 1.6-fold compared to eyes injected with VEGF only (320% ±74 vs 166%±7.8, P=0.03) (FIG. 3).

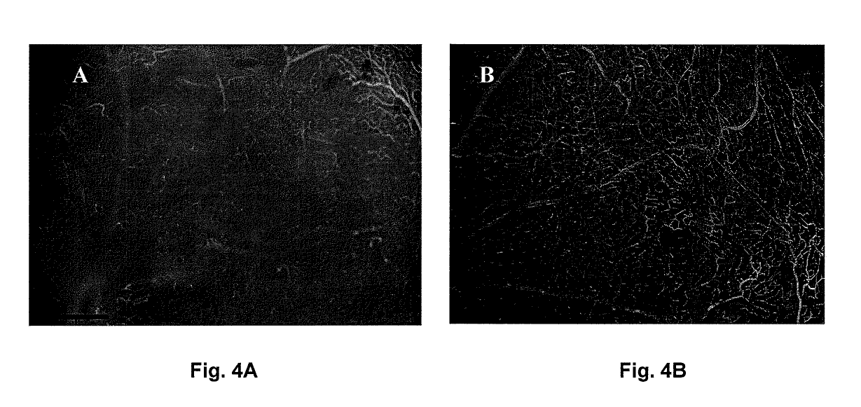

Histological evaluation of the retinal distribution of intravenously injected 20 kDa FITC-conjugated dextran was performed. Evaluation of flat-mount retinas by fluorescent microscopy showed fluorescence diffusely distributed throughout the retinal tissues at both intraluminal and extravascular locations, in the VEGF-injected eyes, while combined treatment with aprotinin demonstrated fluorescein staining more localized to the retinal vasculature (FIGS. 4A and 4B, respectively).

Aprotinin Does Not Reduce VEGF-Induced Leukostasis

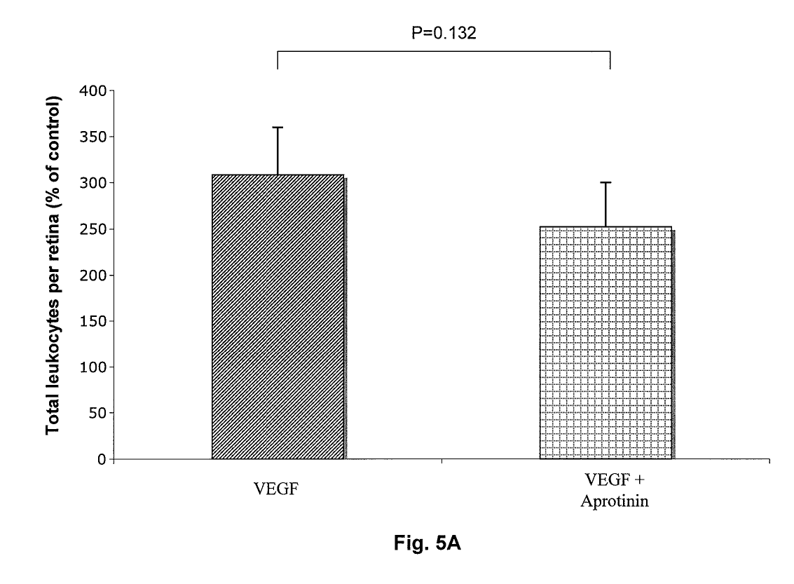

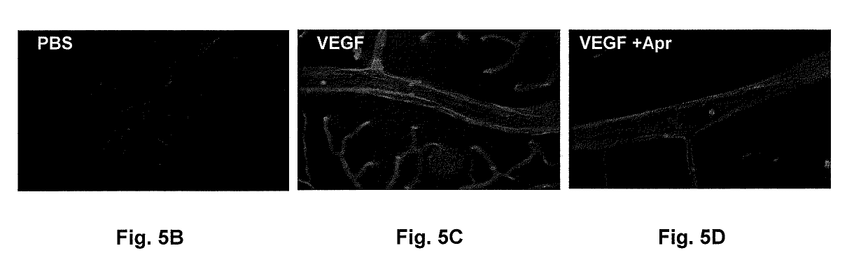

VEGF is a major contributor to increased leukocyte adhesion and concomitant BRB breakdown in the retina. To assess whether aprotinin reduces VEGF-induced retinal vascular permeability through a reduction of leukocyte adhesion, leukocyte adhesion was measured using the Concanavalin A ex vivo retina leukostasis quantization assay. Intravitreal injection of 50 ng of VEGF induces a 3-fold increase in the total number of leukocytes adherent to the retina vessels compared to PBS-injected eyes at 24 hours post injection. When animals were treated with intravenous injections of 50,000 KIU of aprotinin 1 hour before, as well as 8 and 16 hours after, the intravitreal injections of VEGF in one eye and PBS in the contralateral eye, leukocyte adhesion in VEGF-injected eyes (expressed as a percentage of leukocyte adhesion in the contralateral PBS-injected eye) did not differ significantly from leukocyte adhesion in VEGF- and aprotinin-treated eyes (expressed as a percentage of leukocyte adhesion in the contralateral PBS-injected eye) (308% vs 252%, P>0.05) (FIG. 5A). This suggests that the azurocidin inhibitor, aprotinin, does not reduce VEGF-induced BRB breakdown through inhibition of leukostasis. FIG. 5B depicts leukocyte aggregation in control (non-VEGF treated) eyes; FIG. 5C depicts leukocyte aggregation in VEGF-treated eyes; and FIG. 5D depicts leukocyte aggregation in VEGF- and aprotinin-treated eyes.

Taken together, these data suggest that in vivo AZ affects permeability of blood vessels at the BRB by increasing their permeability. Additionally, treatment of eyes displaying AZ-induced or VEGF-induced vascular permeability at the BRB with an AZ inhibitor, such as aprotinin or an antibody against AZ, suppresses vascular leakage, indicating that an AZ inhibitor can be used to treat ocular (and other) disorders involving vascular leakage.

### Example 2

### Direct Effects of VEGF and/or AZ on the BBB

The purpose of this experiment was to investigate the direct effects of VEGF and/or AZ on the BBB. This study used an in vitro model of the BBB in which transendothelial electrical resistance (TEER) in barrier-privileged murine brain microvascular endothelial cells (bEND3) was measured. The results provide support that the direct effects of VEGF and AZ on the endothelium of the BBB, although related, may be distinct.

Materials and Methods

Murine brain microvascular endothelial cells (bEND3) were grown on 0.4 μm fibronectin-coated inserts in 24-well plates (BD Bioscience, San Jose, Calif.). After the cells reached confluency, the cells were stimulated with AZ (AZ, 25 μg/ml), VEGF (100 ng/ml) or both. Electrical resistance was measured at the indicated time-points after the treatment by using a volt-ohmmeter (EVOM, World Precision Instruments, Sarasota, Fla.) with the electrode reproducibly positioned in each well. Transendothelial electrical resistance (TEER) values for each Transwell were calculated by subtracting the electrical resistance of the blank wells (coated filters without cells) prepared and treated as the experimental samples. All experiments were carried out in quadruplicate. The results were expressed as Mean±SEM from n=4 independent experiments.

Results

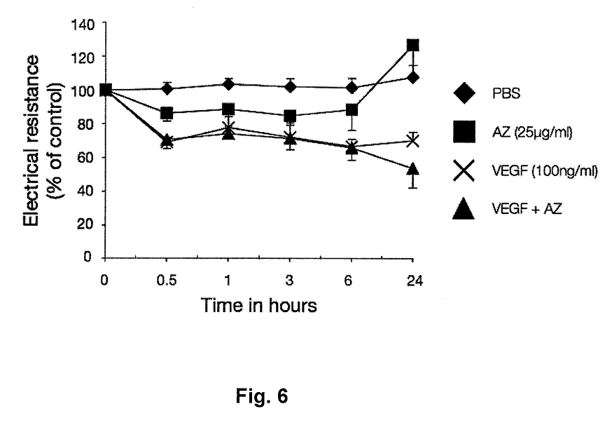

Both VEGF and AZ independently caused a rapid and significant drop of resistance in cultured bEND3 cells within the first 30 minutes after incubation. (FIG. 6) In both VEGF and AZ treated cells, TEER remained at the reduced levels until 6 hours after treatment. Thereafter, TEER in the AZ-treated cells started to recover to normal levels and reached higher values than untreated control, suggesting a rebound phenomenon. In contrast, the VEGF-treated cells showed no sign of recovery in the TEER values, which remained at the reduced levels until 24 hours after treatment.

The ability of AZ-treated cells to recover and the inability of VEGF-treated cells to recover suggests that AZ and VEGF may have independent effects on vascular permeability. Furthermore, treatment of the bEND3 cells with VEGF and AZ together showed significantly lower TEER values compared to VEGF or AZ treated groups alone at 24 hours, suggesting an additive effect of the two compounds.

The initial rapid drop in TEER and the subsequent complete recovery of the BBB-properties after AZ treatment suggest that this molecule may be useful for creating a temporally-defined gate between the intravascular space and the parenchyma of brain and retina, for instance to facilitate drug-delivery to these organs.

### Example 3

### Biologic Action of Aprotinin Applied by Subconjunctival Injection to the Eye

The purpose of this study is to investigate the biologic action of aprotinin when applied as a subconjunctival injection. Example 1 shows that systemic injection of aprotinin reduces injury- or inflammation-induced leakage of retinal vessels. To examine whether aprotinin is biologically active in the back of the eye if it is not made available systemically, the size of CNV was quantified after perfusion with fluorescently labeled dextran in animals treated with subconjunctival injections of the drug or vehicle control.

The subconjunctival mode of delivery was chosen because it is an effective method of delivering drugs, such as dexamethasone or macromolecules, into both the anterior and posterior segments of the eye. Drugs that are able to move from the anterior segment to posterior segment of the eye (e.g., small molecules like steroids) also typically will show an effect in the posterior segment of the eye when injected subconjunctivally. Another mode of local application would be directly using the solution as an eye-drop. However, the use of this drug as an eye-drop is less likely to show biological action in the back of the eye, since there is no depot function for the drug and only small quantities can be used. Furthermore, intravitreal injection of the drug was considered, however, it is not easily possible to perform repeated (i.e., daily) intravitreal injections of the quantities needed for this experiment because of the size of the animal model (such injections may be possible in larger primates, such as humans). Therefore, among the non-systemic modes of delivery, the subconjunctival injection seemed to promise the highest chance of seeing a significant result.

Material and Methods

Thirteen WT mice on C57Bl/6J genetic background (Jackson Laboratories) were used for the study. CNV was induced with a 532 nm laser (Oculight GLx, Iridex, Mountain View, Calif.). Four laser spots (150 mW, 100 μm, 100 ms) were placed in each eye using a slit-lamp delivery system and a cover glass as a contact lens. Occurrence of a bubble immediately after the application of the laser confirmed the rupture of the Bruchs membrane. All animals received daily subconjunctival injections of 20 μl of aprotinin or control (saline). On day 7 after laser injury, the size of the CNV lesions was measured using choroidal flat mounts. Briefly, mice were anesthetized and perfused through the left ventricle with PBS followed by fluorescein labeled dextran (FITC-dextran, MW=2×10⁶; Sigma, St. Louis, Mo.) in 1% gelatin. The eyes were enucleated and fixed in 4% paraformaldehyde for 3 hours. The anterior segment and retina were removed from the eyecup. Four to six relaxing radial incisions were made, and the remaining RPE-choroid-sclera complex was flatmounted with Vectashield Mounting Medium (Vector Laboratories, Burlingame, Calif.) and coverslipped. Pictures of the choroidal flat mounts were taken using an upright fluorescent microscope (Leica). Openlab software (Improvision, Boston, Mass.) was used to measure the magnitude of the hyperfluorescent areas corresponding to the CNV lesions. The average size of CNV lesion in each group was determined.

Results

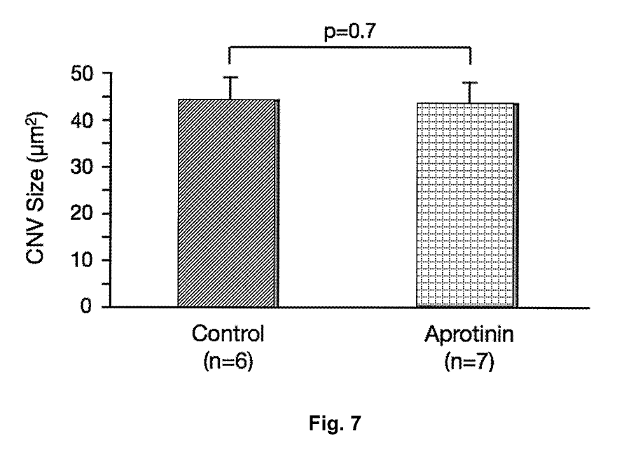

The average CNV size in wild type mice treated with aprotinin or vehicle control by daily subconjunctival injection 7 days after laser induced injury was the same. (FIG. 7) Aprotinin, when applied through daily subconjunctival injections, therefore, does not reduce the amount of leakage or the size of the lesions in the back of the eye. This data suggests that application of the drug in the anterior segment of the eye does not provide a high enough dose in the retinal or choroidal vessels to reduce CNV size. Aprotinin applied by subconjunctival injection may not migrate to or may not otherwise be made available in these areas where the drug's action is desired. Furthermore, it may be that topical application of aprotinin, for instance as an eye drop, would not result in sufficient concentrations of the drug in the back of the eye. These results demonstrate that an effective dose should be delivered to the posterior region of the eye in order to see a biologic effect.

### Example 4

### Diabetic Leukocyte Recruitment In Vivo

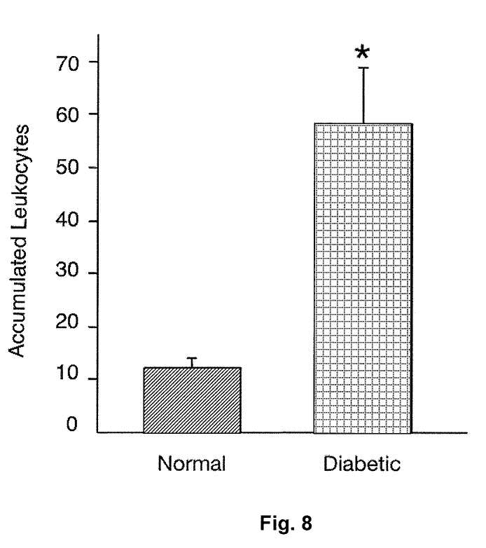

The purpose of this study was to investigate whether AZ plays a role in a chronic inflammatory model, i.e. during diabetes or in aged wild-type mice and ApoE−/−mice. The study used the established method of Scanning Laser Ophthalmoscopy (SLO) in combination with Acridine-Orange Staining of the peripheral blood leukocytes. The inflammatory leukocyte-endothelial interaction in retinal vessels under dynamic conditions of blood flow is able to be visualized using this method. In preliminary experiments, the number of firmly adhering leukocytes in the retinal vessels of normal and diabetic rats (n=6) was quantified, and, consistent with prior reports, higher leukostasis was found in the retinas of diabetic animals (FIG. 8). The data in FIG. 8 represent average values per retina ±SEM, and “*” indicates p<0.01.

### Example 5

### Azurocidin Blockade Reduces Leakage in Diabetic Retinopathy

The purpose of this study was to test whether there is a link between AZ and VEGF in DR, whether AZ mediates vascular leakage in DR, and whether inhibitors of AZ, such as aprotinin, can decrease vascular leakage in DR. Accordingly, AZ's function in diabetic rats was inhibited.

Methods

Induction of Experimental Diabetes

After an overnight fast, Long-Evans rats (Charles River, Wilmington, Mass.), weighing 200-250 g, received single 60 mg/kg intraperitoneal injections of streptozotocin (Sigma, St. Louis, Mo.) in 10 mM citrate buffer (pH 4.5). Control nondiabetic animals received citrate buffer alone. Animals with blood glucose levels greater than 250 mg/dl 24 hours after the streptozotocin injections were considered diabetic. The rats were fed standard laboratory chow and allowed free access to water. Before each experiment, the diabetic state was reconfirmed to be 250 mg/dl or higher.

Results

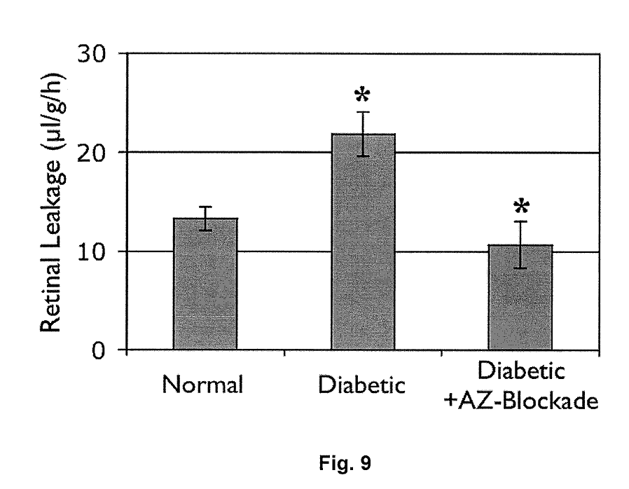

By two weeks after streptozotocin-induced diabetes in the animals, untreated animals showed significant signs of diabetic retinopathy, such as leukostasis and vascular leakage. However, when AZ was blocked with aprotinin (50,000 KIU) in these animals, the vascular leakage was comparable to that of normal animals (FIG. 9). FIG. 9 shows normalized BRB leakage in the retinas of normal and diabetic rats with or without systemic application of aprotinin. Asterisks indicate statistical significance.

The data suggest that AZ is a molecular mediator of organ damage in diabetic retinopathy and potentially other retinopathies with inflammatory leukocyte accumulation and that AZ inhibitors, such as aprotinin, can have a beneficial therapeutic outcome in the treatment of diabetic retinopathy.

### Example 6

### Blockade of AZ Reduces Leakage in a Model of Age-Related Macular Degeneration

The purpose of this study was to test whether AZ may play a role in the pathogenesis of AMD, a chronic neurodegenerative disease, whether AZ mediates vascular leakage in AMD, and whether inhibitors of AZ, such as aprotinin, can decrease vascular leakage in AMD. Accordingly, AZ was inhibited in an experimentally induced model of choroidal neovascularization (CNV).

Methods

Laser-Induced Model of Choroidal Neovascularization (CNV)

Laser-induced CNV is a VEGF-dependent model for exudative changes that occur in AMD. In this model, disruption of an animal's Bruch's membrane by laser results in formation of CNV lesions. In these lesions, neovascular capillaries originate within the choroid and extend through the disrupted Bruch's membrane into the outer nuclear layer of the retina. The formation of these leaky capillaries is mainly attributed to changes in the level of angiogenic growth factors such as VEGF.

To perform the procedure, the pupils were dilated with 1-2 drops of 1% tropicamide. Subsequently, 4-6 photocoagulation lesions were delivered to each eye, using a Coherent argon laser (532 nm wavelength, 50 μm spot size, 0.1 s duration, 120-160 mW; model 920; Coherent, Palo Alto, Calif.) between the retinal vessels in a peripapillary distribution in each fundus, using a slit lamp delivery system and a cover glass as a contact lens. The genesis of a bubble at the time of laser treatment confirmed the rupture of the Bruch's membrane. The extent of laser-induced injury was quantified 2 weeks post injury by fluorescein angiography. Images were captured on a computer (using the IMAGEnet for Windows software, Topcon, Paramus, N.J.). Two masked evaluators performed the grading of the retinal images. A choroidal neovascular membrane is considered to be present if there was early hyperfluorescence followed by later leakage at the site where the laser injury is induced. The eyes were enucleated, preserved, sectioned, and then used for immunohistochemical analysis.

Fluorescein Angiography (FA)

FA is an imaging technique, commonly used for clinical diagnosis of retinal and choroidal vascular leakage and detection of neovascularization. To perform the procedure, animals were injected with 1 ml of a 1:10 diluted 10% solution of sodium fluorescein (Alcon, Fort Worth, Tex.), and the pupils are dilated with 1-2 drops of 1% tropicamide. During FA, images of the retina were captured with a fundus camera (TRC-50VT, Topcon, Paramus, N.J.) and saved electronically. FA was performed before and after the laser coagulation.

Results

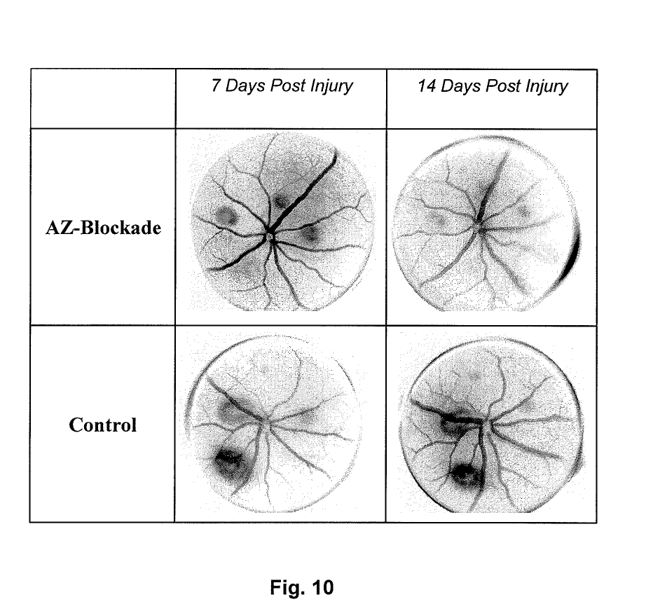

Starting one day prior to the laser injury, mice were given daily injections of the AZ-inhibitor, aprotinin (50,000KIU/day, i.p.). To visualize the amount of leakage and CNV, fluorescein angiography was performed 7 and 14 days after the injury (FIG. 10). In the control mice, substantial leakage was visible after 7 days (FIG. 10, lower left), which slightly increased in size or remained unchanged on the angiograms performed on day 14 post injury (FIG. 10, lower right). In the AZ-inhibitor treated mice, CNV leakage was substantially smaller on day 7 post injury (FIG. 10, upper left), compared to the vehicle treated controls. On day 14, AZ-blockade further reduced lesion size in the treated mice (FIG. 10, upper right). These data suggest that blockade of AZ can effectively reduce choroidal neovascular leakage in a VEGF-dependent model of AMD and that AZ inhibitors, such as aprotinin, can have a beneficial therapeutic outcome in the treatment of AMD.

### Example 7

### Lack of ApoE Exacerbates Leakage in a Model of AMD

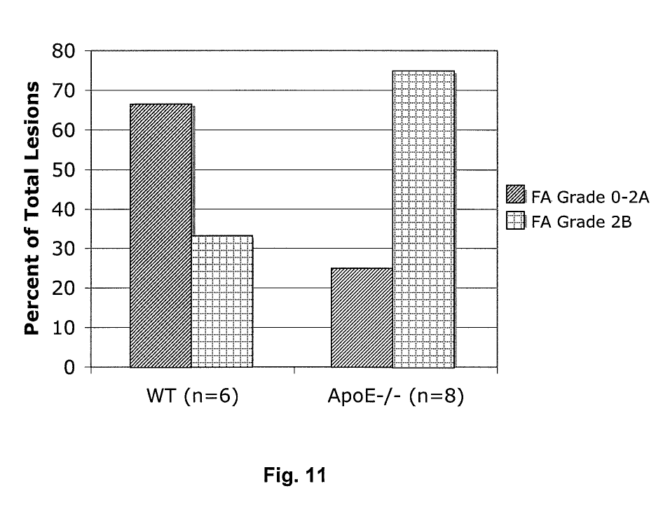

The purpose of this study was to test whether inflammation plays a role in the development of CNV lesions in experimental models of AMD. ApoE is a 34 kDa plasma protein that mediates lipoprotein uptake by receptors such as the LDL receptor. Knowing the established role of ApoE in inflammatory and neurodegenerative diseases and having recently discovered its role in BBB maintenance, it was contemplated that constitutive inflammatory processes may be a cause of BBB defects during aging. To test the relevance of ApoE in the development of choroidal neovascular leakage, ApoE-/- and age-matched wild type mice were used in the laser-induced model of AMD described above. The results show that, in ApoE−/−mice, the percentage of clinically significant lesions (2B) is largely increased (greater than 40%) when compared to age-matched wild type animals (FIG. 11). FIG. 11 shows the percentage of lesions graded as 0, 1 or 2A and lesions graded as 2B (clinically relevant leakage) detected by FA for wild type and for ApoE−/−mice.

To better understand the impact of inflammatory mediators on the BBB/BRB, it is intended to visualize the anatomical microstructures in these vessels by in vivo casting. Additionally, further studies will be conducted to compare the results obtained from wild type mice with that of knockout animals of different ages under different treatment conditions (i.e., AZ, VEGF, and in combination with inhibitors). It is contemplated that the high resolution of SEM will clarify which structures are affected during chronic inflammatory conditions or through inflammatory mediators, such as VEGF or AZ.

### Example 8

### Investigation of AZ Release from Leukocyte-Endothelial Interaction in BBB/BRB Breakdown

To determine whether AZ release due to leukocyte-endothelial interaction underlies BBB/BRB breakdown, AZ or control is applied intravitreally to rat eyes, and BRB permeability is quantified using the EB technique. Aprotinin (30,000 KIU, Trasylol®, Bayer) or a goat polyclonal antibody IgG against AZ (5 μl at 200 μg/ml, Santa Cruz Biotechnology, #SC-20273) is used to block the effect of AZ.

Intravitreous Injections

Intravitreous injections are performed by inserting a 33-gauge double-caliber needle (Ito Corp., Fuji, Japan) into the vitreous approximately 1 mm posterior to the corneal limbus. Insertion and infusion is directly viewed under an operating microscope. Intravitreal injections occasionally can cause injury to the retina or the lens, which can induce inflammation and increase the BRB permeability. Therefore during the intravitreal injections, the tip of the syringe is observed under a microsurgical binocular scope. Any evidence of hemorrhage, edema, or other signs of contact of the syringe with the lens or retina disqualifies the animal from the experiment.

Vascular Casting and SEM

Vascular casting is an established method in the study of the retinal vasculature of rats. This method also has been established for mice, and it is used to elucidate the three dimensional architecture of micro-vessels of wild type and ApoE−/−mice. The changes with age and the response of retinal capillaries to AZ or known mediators of BBB leakage, i.e. VEGF or TNF-α (R&D Systems, Minneapolis, Minn.), will be studied. SEM is used in combination with the vascular casting to study inter-endothelial contact regions in these vessels and to pursue, on a mechanistic level, the cellular and molecular changes that lead to leakage.

To perform the procedures, the animals are anesthetized, and both common carotid arteries are cannulated. A small incision in the jugular veins is made to allow for drainage. The vascular system is perfused with heparinized normal saline solution (500 IU/100 ml), and freshly prepared Mercox CL-2B resin is injected into the cannulated carotid arteries. Subsequently, the eyes are enucleated and left in a warm water bath (56° C.) for 6 hours to allow polymerization and tempering of the resin. The ocular tissues are macerated for 5 days by repeated baths in 20% KOH at room temperature followed by washing with running tap water. The retinal and choroidal vasculature are exposed by microdissection. Casts are again gently washed with running tap water and digested a second time for 2 days to remove residual tissues. The casts are desiccated by freeze drying. The dried vascular casts are impregnated with osmium overnight, then mounted on SEM stubs with double-sided adhesive tape and coated with ion spatter gold-palladium. The casts are examined with a Scanning Electron Microscope (JEOL 7410 F).

It is anticipated that the casts will show that intravitreal injection of AZ will cause BRB breakdown and that antibodies to AZ or aprotinin will block AZ's effects.

### Example 9

### Investigation of whether the BBB/BRB Defect in ApoE−/−Mice is AZ-Mediated

To determine whether the BBB/BRB defect in ApoE−/−mice (i.e., an age-related development of BBB/BRB leakage is AZ-mediated, the AZ blocker, aprotinin, is applied over 2-4 weeks to ApoE−/−mice of various age groups, and their BBB/BRB leakage is quantified at the end of that period using the EB technique. Aprotinin is supplied to the animals via an osmotic reservoir that is placed in the peritoneal cavity of the mice. Even though application of aprotinin for up to 4 weeks should suffice to measure a quantifiable difference, it could be that longer periods of application are necessary. In such a case, the osmotic reservoirs are reloaded with aprotinin, and the mice are treated for an additional 4-6 weeks.

To elucidate whether AZ blockade can prevent the development of accelerated BBB/BRB breakdown, aprotinin is applied for 2-4 weeks to young ApoE−/−mice (i.e., 3-4 weeks old) that have not yet developed the defect. To investigate whether AZ blockade can reverse an established BBB/BRB leakage in mice that have already developed the age-related BBB/BRB defect, the inhibitor is applied for the same period of time to ApoE−/−mice of older age (4-6 months). After application of aprotinin, BBB/BRB leakage in the mice is subsequently quantified.

It is anticipated that application of aprotinin for 2-4 weeks will prevent the BBB/BRB defect in developing ApoE−/−mice. It is possible that AZ blockade with aprotinin also will reverse the defect in older mice.

### Example 10

### Investigation of whether VEGF-Induced BBB/BRB Leakage In Vivo is AZ-Mediated

To further investigate whether VEGF-induced BBB/BRB leakage in vivo is AZ-mediated, wild type mice are intravitreally injected with VEGF (or appropriate controls) with or without an AZ inhibitor, such as aprotinin, and the amount of BRB leakage is quantified. Six hours after intravitreal injection of VEGF (when the VEGF-induced permeability is at its peak), rinsed retinas are dissected from some of the perfused animals to detect AZ using immunohistochemistry or Western Blot. Additionally, in vitro it is investigated whether VEGF can cause the release of AZ from isolated leukocytes. The supernatant of VEGF-treated leukocytes is used to detect AZ using a Western Blot. Briefly, 10-20 ml of human blood is obtained by phlebotomy from healthy volunteers of both genders and all age groups. Leukocyte subtypes (i.e., neutrophils) are isolated using a percoll gradient technique. Then, 1-2×10⁶ leukocytes are incubated for 20 minutes at room temperature with different concentrations (5-500 ng/ml) of human VEGF isoforms (121, 165, and 189) in a total volume of 200-300 μl of 1% FBS-containing PBS. Samples are then centrifuged at 1,500 rpm for 10 minutes, and 150-200 μl of the supernatant is collected for AZ-detection using the Western Blot technique with a commercially available antibody against human AZ (R&D systems).

In is anticipated that VEGF induces the release of AZ from leukocytes, which causes an increase in BBB/BRB permeability.

### Example 11

### Investigation of whether AZ Causes Additional Leukocyte Recruitment to the BBB/BRB, which Perpetuates AZ-Mediated BBB/BRB Degradation

To determine whether AZ causes additional leukocyte recruitment to the BBB/BRB, perpetuating the AZ-mediated BBB/BRB degradation, AZ is injected intravitreally in wild type mice, and SLO is used to perform Acridine Orange Leukocyte Fluorography (AOLF). In addition, rinsed retinal flat mounts are prepared to quantify the number of firmly adhering leukocytes to the endothelium after AZ treatment. It is important that the intravitreal injections are performed properly, because the slightest injury to the retina or the lens may itself cause inflammation and skew the results. Therefore, all experiments involving intravitreal injections include intravitreal injections of vehicle control in the contralateral eye. In case of a vitreous hemorrhage from the injection, the animal is excluded from the study.

Acridine Orange Leukocyte Fluorography with SLO

Leukocyte recruitment in the retina is studied with AOLF. Mice (or rats) are anesthetized, and one day before AOLF a heparin-lock catheter is surgically implanted in the right jugular vein of each animal and externalized to the back of each animal's neck. Each animal's pupils are dilated with 1% tropicamide. Three milligrams per kilogram of acridine orange is injected through the jugular vein catheter at a rate of 1 ml/min, and a focused image of the peripapillary fundus of the left eye is obtained using SLO (Heidelberg, Germany) to evaluate leukostasis in the retina.

Retinal Leukostasis Quantification

The retinal vasculature and adherent leukocytes are imaged with fluorescein-isothiocyanate (FITC)- or rhodamine-coupled Concanavalin A lectin (ConA) (Vector Laboratories, Burlingame, Calif.). To perform the perfusion labeling, animals are anesthetized, the chest cavity is opened, and a 14-gauge perfusion cannula is introduced into the aorta. Drainage is provided from the right atrium. The animals are perfused with 500 ml of PBS/kg body weight (BW) to remove intravascular content and nonadherent leukocytes. Perfusion with ConA (40 μg/mL in PBS [pH 7.4], 5 mg/kg BW) is then performed to label adherent leukocytes and vascular endothelial cells, followed by removal of residual unbound lectin with PBS perfusion. The retinas are carefully removed, fixed with 1% paraformaldehyde, and flat-mounted in a water-based fluorescence-anti-fading medium (Southern Biotech, Birmingham). Images of the retinal microvessels are obtained using epifluorescence microscopy, and the total number of adherent leukocytes per retina is determined.

It is anticipated that AZ injection causes recruitment of neutrophils and macrophages to the BRB within 6-12 hours. This process is self-perpetuating and can cause considerable leakage.

### Example 12

### AZ Blockade Suppresses Retinal Leukocyte Recruitment During Endotoxin-Induced-Uveitis (EIU)

The purpose of this study was to investigate the role of AZ in leukocyte recruitment during uveitis, using the established uveitis model of EIU. It was found that AZ blockade with an AZ inhibitor, such as aprotinin, reduces leukocyte adhesion in retinal vessels and leukocyte extravasation across the BRB. This suggests that AZ inhibitors can be used to treat uveitis and particularly to reduce vascular leakage in uveitis.

Materials & Methods

Endotoxin Induced Uveitis (EIU)

EIU was induced in Lewis rats via footpad injection of 100 μl (1 mg/ml) lipopolysaccharide (LPS). Control animals received an equal volume of saline. The experiments were conducted 24 hours after LPS injection. At this time point, several experimental parameters were assessed.

Reagents

To block AZ, animals received 5 ml of aprotinin (Trasylol®; 50,000KIU). The first injection was given at the same time as the footpad injection, followed by a second injection 8 hours later and a third injection 16 hours later. The control animals received and equal amounts of vehicle control. LPS was purchased in lyophilized form from Sigma (#L6511).

Clinical Evaluation

Using a slit lamp, anesthetized animals were examined in a masked fashion and scored according to the severity of inflammatory response.

Concanavalin A (ConA) Lectin Staining of Adherent Leukocytes

Under deep anesthesia, a rat's chest cavity was opened, and its left ventricle was cannulated to allow perfusion (Ishida (2003) supra). The rat's right atrium was opened to achieve outflow. Twenty milliliters of PBS were perfused to clear erythrocytes and non-sticking leukocytes, followed by 20 ml of FITC-coupled ConA lectin (20 μg/ml in PBS, pH 7.4, total concentration 5 mg/kg; Vector Laboratories, Burlingame, Calif.), which stains adherent leukocytes and the vascular endothelium. The animals were then perfused with PBS alone for 4 minutes to remove excess ConA. The eyes were enucleated, and the retinas were dissected and flatmounted in a water-based fluorescence anti-fading medium (Fluoromount; Southern Biotechnology, Birmingham, Ala.) and imaged by fluorescence microscopy (Leica, FITC filter). Only whole retinas in which the peripheral collecting vessels of the ora serrata were visible were used for analysis. Leukocytes in arteries, veins, and capillaries of each retinal tissue were counted, and the total number of adherent leukocytes per retina was calculated. All analysis was performed in a masked fashion.

Intravitreal Leukocyte Accumulation

To assess leukocyte extravasations into the vitreal cavity during EIU, histological slides of the whole eyes of treated rats were generated and the number of leukocytes in the vitreous cavity of these sections was counted. To do so, whole eyes were placed in 4% paraformaldehyde for fixation. Gradual infiltration of graded alcohols was followed by xylene and finally by liquid paraffin. Paraffin submerged eyes were then cooled to harden. Six-micron sections were cut on a Leica microtome and placed on slides. Sections were stained with hematoxylin and eosin and coverslipped.

Statistics

Statistical differences between experimental groups were analyzed on the Microsoft Excel software program using Student's t-test. Values of P<0.05 were considered statistically significant.

Results

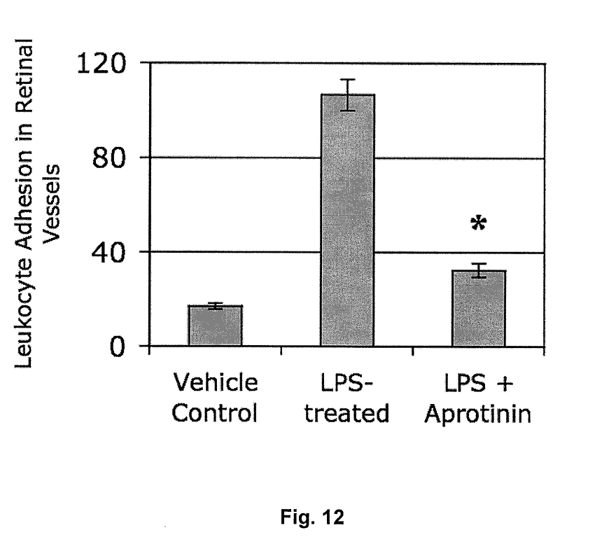

To quantify the inflammatory changes in the posterior chamber, the number of firmly adhering leukocytes in the retinal vessels of normal and EIU rats was counted using the ConA staining technique (FIG. 12). At 24 hours post EIU induction, retinal leukostasis was increased (106.6±6.5 compared to 17±1.2 cells in the normal animals, p<0.01). However, aprotinin treatment significantly reduced retinal leukostasis in EIU animals (32.4±2.9, p<0.01). In FIG. 12, the bars indicate averages of firm leukocyte adhesion in retinal flatmounts (n=6 each group). Error bars indicate the magnitude of the standard error of the mean (SEM). The asterisk indicates p<0.01.

Intravitreal Leukocyte Accumulation

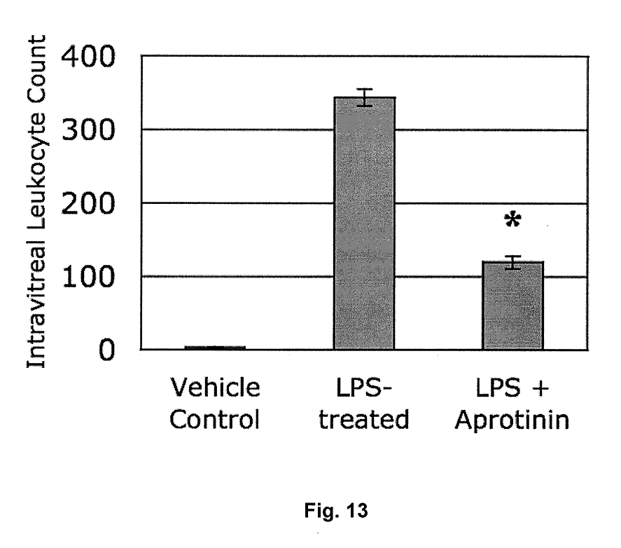

To assess leukocyte extravasations during EIU and elucidate the role of AZ during this process, intravitreal leukocyte accumulation was quantified in eyes from EIU rats treated with aprotinin or control. Whereas normal animals show very few leukocytes (less than 5) in their vitreous cavity, 24 hours post LPS-injection a large number of leukocytes (343.6±11.7, n=6) were found in the vitreous of the EIU animals (FIG. 13). AZ blockade caused a reduction in the number of leukocytes found intravitreally (119.2±8.4, p<0.01). In FIG. 13, the bars show average intravitreal leukocyte numbers obtained from histologic sections of the whole eye of rats. Error bars indicate the magnitude of SEM, and the asterisk indicates p<0.01.

Role of Adhesion Molecules in Uveitis

The importance of a variety of adhesion molecules to the outcome of experimental EIU has been previously explored. As a result, P- and E-selectin, β₂-integrins, and ICAM-1 have been found to be involved in the ocular inflammatory responses to endotoxin. Blockade of P- and E-selectin results in a lower ICAM-1 expression in the eyes of EIU animals, suggesting that selectin blockade may influence the course of intraocular inflammation. Another recently uncovered target for treatment of endotoxin-induced inflammation is Lectin-like oxidized LDL receptor-1 (LOX-1), an adhesion molecule involved in leukocyte recruitment. This adhesion molecule is also expressed in eyes with posterior uveitis and are likely regulated by cytokines such as TNF-α. Blockade or lack of each adhesion molecule listed above does not alone abolish leukocyte infiltration to the eye, suggesting that other molecules with a functional overlap participate in this process.

Firm adhesion marks the beginning of leukocyte extravasation across the BBB in inflammatory diseases of the CNS, such as experimental autoimmune encephalomyelitis (EAE) or EIU. Since AZ is known to cause arrest of certain leukocyte subtypes in vitro, it may play a role in leukocyte recruitment during EIU. Our results suggest that blockade of AZ by aprotinin can significantly reduce firm leukocyte adhesion in the retinal vessels and leukocyte extravasation across the BRB. This suggests that AZ inhibitors can be used to treat uveitis, and particularly to reduce vascular leakage in uveitis.

Equivalents

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

Incorporation by Reference

The entire disclosure of each of the patent documents and scientific publications disclosed hereinabove is expressly incorporated herein by reference for all purposes.

What is claimed is:

1. A method for reducing vascular permeability of an ocular blood vessel, the method comprising administering to a posterior region of a mammal's eye an amount of an azurocidin inhibitor effective to reduce vascular permeability of an ocular blood vessel located in the posterior region of the eye.

2. The method of claim 1 wherein the azurocidin inhibitor comprises a Kunitz type protease inhibitor.

3. The method of claim 2 wherein the Kunitz type protease inhibitor is selected from the group consisting of aprotinin, pancreatic trypsin inhibitor, WFIKKN protein, broad spectrum Kunitz type serine protease inhibitor secreted by Ancylostoma ceylanicum, potato serine protease inhibitor, trypstatin, bikunin, BbKI found in Bauhinia bauhiniodes seeds, and members of the I-α-I family of Inter-α-inhibitors.

4. The method of claim 2 wherein the Kunitz type protease inhibitor comprises aprotinin.

5. The method of claim 1 wherein the azurocidin inhibitor comprises heparin or a heparin-related molecule.

6. The method of claim 5 wherein the heparin-related molecule comprises a heparin-like glycosaminoglycan or a heparin-like oligosaccharide.

7. The method of claim 1 wherein the azurocidin inhibitor comprises an antibody.

8. The method of claim 1 wherein the azurocidin inhibitor comprises an aptamer.

9. The method of claim 1 wherein the azurocidin inhibitor comprises an siRNA.

10. A method for ameliorating a symptom of an eye condition comprising administering to a mammal an amount of azurocidin inhibitor effective to ameliorate a symptom of the eye condition.

11. The method of claim 10 wherein the eye condition comprises diabetic retinopathy.

12. The method of claim 10 wherein the eye condition comprises macular edema.

13. The method of claim 10 wherein the eye condition comprises age-related macular degeneration.

14. The method of claim 10 wherein the eye condition comprises uveitis.

15. The method of claim 10 wherein the eye condition comprises elevated blood pressure.

16. The method of claim 10 wherein the eye condition comprises an ischemic retinopathy.

17. The method of claim 10 wherein the eye condition comprises choroidal neovascularization.

18. The method of claim 10 wherein the symptom comprises vascular leakage.

19. The method of claim 10 wherein the azurocidin inhibitor comprises a Kunitz type protease inhibitor.

20. The method of claim 19 wherein the Kunitz type protease inhibitor is selected from the group consisting of aprotinin, pancreatic trypsin inhibitor, WFIKKN protein, broad spectrum Kunitz type serine protease inhibitor secreted by Ancylostoma ceylanicum, potato serine protease inhibitor, trypstatin, bikunin, BbKI found in Bauhinia bauhiniodes seeds, and members of the I-α-I family of Inter-α-inhibitors.

21. The method of claim 19 wherein the Kunitz type protease inhibitor comprises aprotinin.

22. The method of claim 10 wherein the azurocidin inhibitor comprises heparin or a heparin-related molecule.

23. The method of claim 22 wherein the heparin-related molecule comprises a heparin-like glycosaminoglycan or a heparin-like oligosaccharide.

24. The method of claim 10 wherein the azurocidin inhibitor comprises an antibody.

25. The method of claim 10 wherein the azurocidin inhibitor comprises an aptamer.

26. The method of claim 10 wherein the azurocidin inhibitor comprises an siRNA.

27. The method of claim 10 wherein the azurocidin inhibitor is administered locally or systemically.

28. The method of claim 10 wherein the azurocidin inhibitor is administered by intraocular, intravitreal, subconjunctival, or transcleral administration.

29. The method of claim 10 wherein the azurocidin inhibitor is administered by intravitreal administration.

30. The method of claim 10 wherein the azurocidin inhibitor is administered by at least one of an implant, iontophoresis, and encapsulated microbubbles.
